SEQUENCE LISTING (7 sequences)

SEQ ID NO: 1 - protein, 17 aa, Artificial Sequence
  D-Peptide
NEMYLWHWQYPQHLRVG

SEQ ID NO: 2 - protein, 16 aa, Artificial Sequence
  D-Peptide
DGGYQILFKIPGGHIH

SEQ ID NO: 3 - protein, 21 aa, Artificial Sequence
  D-Peptide
NEMYLWHWQYPQHLRVRRRRR

SEQ ID NO: 4 - protein, 21 aa, Artificial Sequence
  D-Peptide
NELYLWHWQYPQHLRVRRRRR

SEQ ID NO: 5 - protein, 21 aa, Artificial Sequence
  D-Peptide
DGGYQILFKIPGGHIHRRRRR

SEQ ID NO: 6 - protein, 21 aa, Artificial Sequence
  D-Peptide
RSSYQILFRIPSSHIHRRRRR

SEQ ID NO: 7 - protein, 18 aa, Artificial Sequence
  D-Peptide
YQILFRIPSSHIHRRRRR

(19) United States
(12) Patent Application Publication
(10) Pub. No.: US 2025/0326795 A1
(43) Pub. Date: Oct. 23, 2025

(54) USE OF D-ENANTIOMERIC PEPTIDE LIGANDS OF MONOMERIC TAU FOR THE THERAPY OF VARIOUS TAUOPATHIES

(71) Applicant: PRIAVOID GMBH, Duesseldorf (DE)

(72) Inventors: Dieter WILLBOLD, Juelich (DE); Jeannine MOHRLUEDER, Erkelenz (DE); Marc SEVENICH, Zuelpich (DE); Karoline Bianka SANTUR-DABOW, Koeln (DE); Tim ALTENDORF, Wuerselen (DE)

(21) Appl. No.: 18/283,088

(22) PCT Filed: Mar. 25, 2022
(86) PCT No.: PCT/EP2022/057923

§ 371 Date: Apr. 3, 2024

(30) Foreign Application Priority Data
Mar. 25, 2021 (DE) 10 2021 107 546.7

(51) Int. Cl. C07K 7/08; A61K 38/00

(52) U.S. Cl. CPC C07K 7/08; A61K 38/00

(57) ABSTRACT

The invention relates to a peptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7 and homologues, fragments and parts thereof, as well as to such a peptide for use in the treatment of tauopathies.

### SEQUENCE LISTING

The instant application contains a Sequence Listing which has been submitted electronically in ASCII format and is hereby incorporated by reference in its entirety. Said ASCII copy, created on Apr. 1, 2024 is named P50813_SL_trans and is 1,806 bytes in size.

### BACKGROUND OF THE INVENTION

### 1. Field of the Invention

The invention relates to a peptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7, homologues, fragments and parts thereof, as well as to such a peptide for use in the treatment of tauopathies.

### 2. Discussion of Background Information

Alzheimer's dementia (AD) is the most common neurodegenerative disease worldwide, with an estimated portion of almost two-thirds, which corresponds to ca. 27 million people. It is assigned to a heterogeneous group of different tauopathies, which are symptomatically characterised by a progressive dementia. The intraneuronal deposits that thereby occur in the brain, which contain amyloid structures of the hyperphosphorylated tau protein, usually correlate with the extent of cognitive deficits in the course of the disease.

As a neuronal microtubule-associated protein, tau is significantly involved in the assembly and stabilisation of microtubules. Hyperphosphorylation of tau triggers its misfolding and aggregation up to the formation of so-called neurofibrillary tangles, which are a neuropathological hallmark of most tauopathies. In the adult brain, six tau isoforms are expressed by alternative splicing, which are between 352 and 441 amino acids long. Depending on the composition of isoforms present, different misconformations can be formed that are specific to the tau pathology at hand.

Toxic, self-replicating and propagating tau aggregates are a hallmark of various tauopathies. In these pathologies, there is a characteristic prion-like proliferation of misfolded tau aggregates in the CNS, which is associated with a neurodegeneration of the affected brain areas. Several studies were able to demonstrate the uptake of tau aggregates from cells, “seeding” effects (aggregates that induce the conversion of monomeric to misfolded, oligomeric or fibrillar tau as templates), as well as tau aggregate transfer between cell cultures. The pathways of the tau-associated pathology herein reflect neuroanatomically connected brain regions.

To date, no active substance or drug exists that is effective against the causes of AD. The drugs that have been used and approved so far alleviate some of the symptoms that occur in Alzheimer's dementia. However, they are not able to slow down the progress of the disease or bring about a cure. Some substances exist that have shown success in animal studies in the prevention, but not (necessarily) the treatment of AD.

However, five drugs (Aricept, Exelon, Razadyne, Donepezil and Namenda) are available that relieve the symptoms of the disease and make everyday life easier for those affected.

Frontotemporal dementia is also not treatable causally, so that here too the course of the disease can neither be slowed down nor stopped. Primarily, antidepressants are used, which can have a symptom-relieving effect.

For the progressive supranuclear palsy, there is as yet no possibility of stopping or slowing down the pathology. Some symptoms do not respond to any medication. Antidepressants (e.g. Prozac, Elavil and Tofranil) are used to alleviate other symptoms, and special glasses and walking aids are used to make everyday life easier.

Subacute sclerosing panencephalitis caused by a measles infection cannot be cured at present. Antiviral therapeutics (isoprinosine and ribavirin), as well as immunomodulatory substances (interferon alpha) can stop the progress of the disease and increase the life expectancy of patients. However, the side effects of long-term treatment are up to now unknown.

So far, there is no drug available for corticobasal degeneration that slows down or stops the progression of the disease. The symptoms of corticobasal degeneration are usually resistant to treatment. The substance clonazepam is used to treat myoclonia. physio and speech therapies can help with coping with everyday life.

In summary, a causative and significantly life-prolonging therapy is not yet available for most, if not all, tauopathies and is urgently needed.

The object of the present invention was therefore the development of new chemical entities that can minimize the formation of new aggregates, as well as disassemble existing toxic tau aggregates into native functional tau monomers, and thus their therapeutic use in various tauopathies is possible.

The chemical entity to be used in therapy or its variants should bind as affinely and specifically as possible to the native, endogenous, monomeric tau protein and thus stabilise it. The equilibirium between misfolded and natively folded tau conformation is thus shifted in favour of the latter. Thus, in ideal circumstances, already existing tau oligomers and fibrils can be broken down into their monomeric components and thus eliminated.

### SUMMARY OF THE INVENTION

This object was solved by a peptide according to claim 1, in particular by a peptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7 as well as homologues, fragments and parts thereof.

Further preferred embodiments are defined in the dependent claims.

Hereinafter, the term “comprising” shall also include “consisting of”.

The peptides of SEQ ID NO: 1, SEQ ID NO: 2, SEQ ID NO: 3, SEQ ID NO: 4, SEQ ID NO: 5, SEQ ID NO: 6 or SEQ ID NO: 7 were found with the aid of an optimised mirror image phage display selection.

It should be noted that, in addition to the selection of specific monomer binders indicated here, the mirror image phage display method can of course also be used to find specific oligomer binders or even to find ligands towards other species occurring in protein misfolding disease.

In the mirror image phage display, for example, a recombinant library of randomized peptide sequences presented at the gp3 protein of the M13 phage and encoded in its genome is selected against the exact mirror image (D-enantiomer) of a naturally occurring L-enantiomeric target molecule (e.g. tau).

The peptide sequence is advantageously presented at the N-terminus of the gp3 protein of the M13 phage and is present in encoded form in its genome.

The gp3 molecule, also called gene product 3, is a protein that sits in the phage's envelope and is required for contact with the host cell.

The DNA sequence of the p3 gene of a selected phage is linked to the DNA sequence that contains the genetic information about the corresponding peptide sequence on the gp3 molecule, allowing it to be sequenced. After sequencing, the genomic sequence can be transcribed into an amino acid sequence and synthesised as a D-enantiomeric peptide that binds to the physiological L-enantiomeric form of the target molecule (e.g. tau).

The totality of phages that present different peptides as fusion protein with gp3 on their surface is referred to in the following as the phage library. The corresponding peptides represent the biomolecules to be selected in the experiment.

So-called panning rounds can be carried out, e.g. three rounds. Therein, the phage library is brought into contact with a fixed target molecule, also called bait, and binding phages are isolated from the billion-fold background of other, non-binding phages.

Exemplary, the amount of phages that preferentially bind to oligomeric or fibrillar species of tau is reduced by not offering these very species as bait. Phages that show an increased affinity for tau oligomers and fibrils can be removed from the phage pool in this way, so that, e.g., tau monomer-specific phages accumulate. The method can, of course, be adapted in an analogous way in order to specifically identify tau oligomer-binding ligands and peptides.

Furthermore, in order to reduce enrichment of phages with affinity for plastic, BSA or streptavidin, according to the invention different substrate surfaces are used in preferably all panning rounds. In this case, the substrate surface is defined as a combination of the substrate used (plastic plate, in this case polystyrene with a streptavidin matrix) and the blocking or quenching agents used. In the successive selection rounds, the selection pressure is successively increased.

For this purpose, while the concentration of the target molecule (e.g. monomeric tau) remains stable, the number of washing steps after phage incubation is continuously increased from the second selection round onwards in order to remove non-tau-monomer-affine phages.

Furthermore, a different substrate surface is selected in each selection round of the phage display by using different agents to block the surface after immobilisation of the target molecule on the substrate (e.g. BSA, milk powder, no blocking). Exemplary, it is possible to switch between an in addition to the biotin-treated milk powder-blocked surface in round 1, a BSA-blocked surface in round 2 and a milk powder-treated surface in round 3.

The switching between different substrate surfaces increases the specificity for the target molecule, or bait, towards the surface. There is also a reduction of the ligands that bind non-specifically to plastic surfaces or the blocking agent.

Parallel to the actual phage display selection, exemplary control selections can be carried out, which are identical to the main selection in terms of execution—with the important difference that no bait is used here. A data analysis of the sequences that result from the control selections allows the identification of peptides that accumulate during the selection even without bait and are thus irrelevant for all subsequent steps.

The method is thus characterized by the following steps:
a) Providing an immobilized bait on a substrate surface.
b) Contacting the immobilized molecule acting as bait with a solution containing a library of molecules.
c) Contacting the immobilized bait loaded with the molecules with a wash solution.
d) Separation and multiplication of the molecules still bound to the bait after contacting the immobilized bait loaded with the molecules with washing solution.
e) Repetition of the above steps, wherein with each repetition a different substrate is used,
f) identifying the sequence of the molecules remaining on the bait after the repetition.

A different substrate is used, for example, by the changing of the type of substrate and/or its blocking or non-blocking by means of reagents.

As bait a molecule selected from the group consisting of proteins, peptides, RNA, DNA, m-RNA and chemical compounds is used. In particular, monomeric tau protein is used as bait in the present case.

As surface or substrate on which the bait is immobilized, for example, a component from the group consisting of microtiter plates, magnetic particles, agarose beads or Sepharose beads is used.

The bait according to point a) is therefore a compound to which the biomolecule to be selected is to be bound. It is fixed to a first surface according to methods known in the prior art. Exemplary, but not limiting, as baits proteins, peptides, RNA or DNA molecules may be mentioned, in particular tau monomers. As possible surfaces exemplarily microtiter plates, magnetic particles, agarose beads or Sepharose beads may be used.

The surface with the immobilised bait can subsequently be quenched, whereby the functional groups of the substrate are inactivated. In addition, blocking of the free areas remaining on the substrate can be carried out with suitable reagents.

In the second step b), the immobilized bait is brought into contact with a randomized library of molecules-specifically biomolecules. These biomolecules compete for binding to the bait. The randomized library is a mixture of very many, for example 1012, but also 104 or only 100 different molecules in a mixture. Such a library can, for example, consist of peptides, proteins, DNA, RNA or m-RNA, which are present bound to certain vehicles and which can bind to bait. As vehicles may be considered, for example, phages, polysomes or bacterial surfaces. The library may consist of artificial components or components isolated from nature, or a mixture of both. As artificial in the sense of the invention are to be understood, for example, compounds produced from oligonucleotide synthesis.

It is possible to contact the immobilized bait loaded with biomolecules with a washing substance in step c). For this purpose, a washing step is carried out in step c), in which a buffer solution is brought into contact or rinsed with the immobilized baits. This means that the solution containing the library of biomolecules is preferably repeatedly replaced by a possibly similar or identical solution. Thus, those library molecules are removed which dissociate off less rapidly from the immobilized baits than other library molecules. The speed of the dissociation reaction of the binding library molecules is mainly determined by the different dissociation constants (especially the k_{off} values) of the individual molecules. Those with a small k_{off} value statistically remain bound to the immobilized bait the longest and thus have a lower statistical probability of being washed away by the wash buffer. The liquid containing the wash buffer is preferably aqueous and may contain a pH buffer. Optional components of the solution for the washing step may be salts, detergents or reducing agents.

After the specificity washing step in step c), the bound biomolecules are separated from the bait and multiplied in step d).

The separation in step d) is done, e.g., by elution of phages from the bait. The separation can be achieved, for example, by changing the pH, heating or changing, in particular increasing the salt concentration. In the subsequent multiplication of the library molecules still remaining on the bait, the phage particles obtained, for example, according to steps a) to c) can be introduced into cells and multiplied.

In step e), the concentration of the selected biomolecules is increased in the solution which is added to the bait after step a). Preferably, 3 to 6 selection rounds which comprise the steps a) to e) are performed. However, 1 to 10 or 1 to 20 repetitions may also be carried out. Also, the therewith preferably done increase in the competitor concentration in step c) leads to an improved selection with increasing number of cycles.

A particularly relevant mirror image phage display provides for N-terminally biotinylated D-enantiomeric tau monomer in step a), a recombinant phage library in step b) as well as a buffer solution in step c) in addition to tau monomer as bait. An elution as a separation step is carried out, e.g., by lowering the pH value as a separation step and phage amplification in bacterial cells as multiplication.

In this way, seven D-enantiomeric peptides that bind specifically to tau monomers were selected.

SEQ ID NO 1:
TF2D-1 (free N-terminus, amidated C-terminus):
Nemylwhwqypqhlrvg
SEQ ID NO 2:
TF2D-5 (free N-terminus, amidated C-terminus):
dggyqilfkipgghih
SEQ ID NO 3:
TF2D-1a (free N-terminus, amidated C-terminus):
nemylwhwqypqhlrvrrr
SEQ ID NO 4:
TF2D-1b (free N-terminus; amidated C-terminus):
nelylwhwqypqhlrvrrrrr
SEQ ID NO 5:
TF2D-5a (free N-terminus; amidated C-terminus):
dggyqilfkipgghihrrrrr
SEQ ID NO 6:
TF2D-5b (free N-terminus, amidated C-terminus):
rssyqilfripsshihrrrrr
SEQ ID NO 7:
TF2D-5c (free N-terminus, amidated C-terminus):
yqilfripsshihrrrrr

The present invention may also relate to further peptides that can be identified using the method disclosed above.

The peptides according to SEQ ID NO: 1 to 7 can be used as a possible drug against tauopathies, such as Alzheimer's dementia, due to the specific binding to tau monomers.

The object according to the invention is also solved by a peptide containing homologues, fragments and parts of the amino acid sequence according to SEQ ID NO: 1 to 7.

By tau peptide or tau protein is preferably understood here the human tau peptide or tau protein.

For the purposes of the invention, “homologous sequences” or “homologous” means that an amino acid sequence has an identity with one of the above-mentioned amino acid sequences of the monomers of at least 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%. Preferred here are 80% and 90%. Instead of the term “identity”, the terms “homolog” or “homology” are used synonymously in the present description. The identity between two nucleic acid sequences or polypeptide sequences is calculated by comparison with the aid of the program BESTFIT based on the algorithm of Smith, T. F. and Waterman, M. S (Adv. Appl. Math. 2:482-489 (1981)) with setting the following parameters for amino acids: gap creation penalty: 8 and gap extension penalty: 2; and the following parameters for nucleic acids: gap creation penalty: 50 and gap extension penalty: 3.

Preferably, the identity between two nucleic acid sequences or polypeptide sequences is defined by the identity of the nucleic acid sequence/polypeptide sequence over the respective entire sequence length as calculated by comparison with the aid of the program GAP based on the algorithm of Needleman, S. B. and Wunsch, C. D. (J. Mol. Biol. 48:443-453) with setting the following parameters for amino acids: Gap creation penalty: 8 and Gap extension penalty: 2; and the following parameters for nucleic acids Gap creation penalty: 50 and Gap extension penalty: 3.

Two amino acid sequences are identical for the purposes of the present invention if they have the same amino acid sequence.

Under homologues, in one variant, the peptide sequences according to the invention are of, which differ from the sequences indicated by up to two or three amino acids.

Furthermore, sequences containing the above sequences can also be used as peptides.

The peptides according to the invention are further preferably characterised in that an acid amide group (CONH₂ group) or a COH group, COCl group, COBr group, CONH-alkyl residue or a CONH-alkyl-amine residue is present at the free C-terminus instead of the carboxyl group, or else the peptide is cyclised.

In this way, in a particularly advantageous manner the object is additionally solved that a peptide without a negative charge at the C-terminus is provided. By this it is advantageously effected that this can bind to the target molecule with a higher affinity than a peptide which has a carboxyl group at the free C-terminus. Peptides with a free, unmodified carboxyl group have a negative charge at this end in the physiological state.

In one embodiment of the invention, the peptides according to the invention in the physiological state, in particular at pH 6-8, in particular 6.5-7.5, in particular at pH 6, 0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9 or pH 8.0 are modified such that the C-terminus does not carry a negative charge, but instead is neutral or has one or more positive charges.

In one embodiment, the peptide is characterized in that an acid amide group is present at the free C-terminus in place of the carboxyl group. Thus, instead of the carboxyl group (—COOH group), an acid amide group (—CONH₂ group) is arranged at the C-terminus.

The peptide is thus particularly advantageously amidated at the free C-terminus and not modified at the free N-terminus.

The peptide is thus particularly advantageously amidated at the free C-terminus and not modified at the free N-terminus.

By this the further object of providing a peptide without negative excess charge, which can bind more affinely to the target molecule and is obtainable in a simple manner is solved particularly advantageously.

In a further embodiment of the invention, the following further groups are present in place of the carboxyl group: COH, COCl, COBr, CONH-alkyl residue, CONH-alkyl-amine residue (net positive charge), etc., although one does not limit thereto, as long as the technical teaching of the main claim is followed.

In a further preferred embodiment of the invention, therefore, the affinity of the bond of the peptides modified according to the invention without negative charge at the C-terminus, compared to linear peptides with negative charge at the C-terminus but otherwise the same amino acid sequence, is increased by 1%, 2, 3, 4, 5, 6, 7, 8, 9, in particular 10%, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, in particular 100%, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, in particular 200%, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, in particular 300%, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, in particular 400%, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, advantageously even 500%, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, particularly advantageously 600%, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, particularly advantageously 700%, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, also particularly advantageously 800%, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, also particularly advantageously 900%, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or even by 1000%, or even by 10000%, or even by up to 100000% or 1000000%, wherein any intermediate value can be assumed.

This is indicated by a correspondingly lowered K_{D} value. The K_{D} value as a measure for the affinity of the binding of a modified peptide to tau monomer is in comparison to a linear binding peptide with negative charge at the free C-terminus lowered by 1%, 2, 3, 4, 5, 6, 7, 8, 9, especially 10%, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, in particular 99.1, 99.2, 99.3, 99.4, 99.5%, 99.6, 99.7, 99.8, 99.9 up to 99.99 or even 99.999%, wherein any intermediate value can be assumed.

The peptide according to the invention is further preferably characterized in that it contains 2, 3, 4, 5, 6, 7, 8, 9, 10 or more copies of the sequences with SEQ ID NO: 1, SEQ ID NO: 2, SEQ ID NO: 3, SEQ ID NO: 4, SEQ ID NO: 5, SEQ ID NO: 6 and/or SEQ ID NO: 7.

Variants are also conceivable wherein the peptide contains 11, 12, 13, 14, 15, 16, 17, 18, 19, 20 or more peptides having SEQ ID NO: 1, SEQ ID NO: 2, SEQ ID NO: 3, SEQ ID NO: 4, SEQ ID NO: 5, SEQ ID NO: 6 and/or SEQ ID NO: 7.

In particular preferred are dimers of the sequences with SEQ ID NO: 1, SEQ ID NO: 2, SEQ ID NO: 3, SEQ ID NO: 4, SEQ ID NO: 5, SEQ ID NO: 6 and/or SEQ ID NO: 7, wherein the two monomers of the dimer are peptides with the same SEQ ID or with a different SEQ ID.

The peptides according to the invention are further preferably characterized in that they consist essentially of D-amino acids.

For the purposes of the present invention, the term “essentially of D-enantiomeric amino acids” means that the peptides to be used according to the invention are composed of at least 50%, 55%, 60%, 65%, 70% preferably 75%, 80%, particularly preferably 85%, 90%, 95%, in particular 96%, 97%, 98%, 99%, 100% of D-enantiomeric amino acids.

The peptide according to the invention is further preferably characterized in that it consists of an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7.

The peptide according to the invention is further preferably characterised in that the peptide is linked to a further substance.

For the purposes of the invention, the linkage is a chemical bond as defined in Römpp Chemie Lexikon, 9th edition, volume 1, page 650 ff, Georg Thieme Verlag Stuttgart, preferably a principal valence bond, in particular a covalent bond.

In one variant, the substances are medicinal products or active substances, defined according to the Medicinal Products Act (Arzneimittelgesetz) § 2 or. § 4 (19), as of September 2012. In an alternative, active substances are therapeutically active substances which are used as medicinally active substances. Preferably, anti-inflammatory substances are used.

In another alternative, the substances are compounds that enhance the effect of the peptides.

In another alternative, the substances are compounds which improve the solubility of the peptides and/or the passage of the blood-brain barrier.

In an alternative, the peptides according to the invention have any combination of at least two or more features of the variants, embodiments and/or alternatives described above.

The peptide according to the invention is further preferably characterized in that several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are covalently or non-covalently linked to each other.

A covalent linkage or linkage of the peptide units is present in the sense of the invention if the peptides are linearly linked to each other head to head, tail to tail or head to tail, with or without interposed linker or linker groups.

The peptide according to the invention is further preferably characterized in that several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other without linker, i.e. directly, or are linked to each other with a linker group.

A non-covalent linkage within the meaning of the invention is present if the peptides are linked to each other, for example, via biotin and streptavidin, in particular streptavidin tetramer.

The peptide according to the invention is further preferably characterized in that several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other in a linear or branched manner.

In one variation of the present invention, the peptides may be linearly linked to each other, in particular as described above. In another variant, the peptides are linked to each other in a branched manner to form the peptide of the invention.

According to the invention, a branched peptide may be a dendrimer in which the monomers are covalently or non-covalently linked to each other.

Alternatively, the peptides may also be linked to a platform molecule (such as PEG or sugar) and thus form a branched peptide.

Alternatively, combinations of these options are also possible.

In one embodiment of the invention, the affinity of the binding of the peptides is defined by the dissociation constant (K_{D} value).

The dissociation constant (K_{D} value) of a peptide according to the invention is thereby advantageously lowered in an advantageous embodiment of the invention. This is associated with improved properties of the peptides according to the invention, such as higher affinity of binding and higher effectiveness of degradation and/or prevention of the formation of toxic tau oligomers or aggregates.

In one embodiment of the invention, such peptides are used which binds to a tau monomer with a dissociation constant (K_{D} value) of at most 500 μM, preferably 250, 100, 50 μM, particularly preferably 25, 10, 1 μM, particularly preferably with a dissociation constant (K_{D} value) of at most 500 nM, 250, 100, 50, particularly preferably 25, 10, 1 nM, 500 pM, 100, 50, 25, 20, 15, 10, 9, 8, 7, 6, 5, 4, 3, 2, 1 pM to sub-pM, wherein any intermediate value can be assumed.

The peptides according to the invention are further preferably, characterized in that they bind to the tau protein, preferably the natively folded tau protein, with a K_{D} of less than 1 pM.

The peptides can be produced, for example, via chemical synthesis or peptide synthesis.

In a further variant, the peptide according to the invention is for inhibiting or preventing the formation of tau peptide oligomers and/or tau peptide aggregates.

In a further variant, the peptide according to the invention is for the detoxification of tau peptide oligomers and/or tau peptide aggregates.

The peptides according to the invention detoxify the tau-peptide oligomers and/or tau-peptide aggregates or polymers as well as fibrils formed therefrom preferably by not binding to them but by binding to tau monomers and by shifting the equilibrium leading to the reduction of the tau oligomers and thus converting them into non-toxic compounds. Accordingly, also a subject matter of the present invention is a method for the detoxification of the tau oligomers, aggregates or fibrils formed therefrom.

The inhibition or the prevention of the formation of tau-peptide oligomers and/or tau-peptide aggregates, or the detoxification of the tau-peptide oligomers and/or tau-peptide aggregates, may occur in vitro or in vivo.

The present invention also relates to a peptide according to any of the preceding claims for use in the treatment of tauopathies.

The present invention also relates to a peptide according to any one of the preceding claims, in particular for use in the treatment of Alzheimer's dementia (Alzheimer).

### BRIEF DESCRIPTION OF THE DRAWINGS

In the drawings,

FIG. 1 is a graph illustrating that TF2D-1 and TF2D-5 inhibit tau aggregation;

FIG. 2a is a graph illustrating that TF2D-5 binds with high affinity to tau protein;

FIG. 2b is a graph illustrating that TF2D-5 and TF2D-5a bind with high affinity to tau protein;

FIG. 3 is a graph illustrating that TF2D-5 inhibits tau aggregation, analyzed by ThT and AFM analysis;

FIG. 4 is a graph illustrating that TF2D-5 and TF2D-5a affinity is sequence specific;

FIG. 5 is a graph illustrating that TF2D-5a inhibits the aggregation of tau in cells; and

FIG. 6 is a graph illustrating that TF2D-5 and TF2D-5a are stable in different body fluids.

### DETAILED DESCRIPTION OF EMBODIMENTS OF THE INVENTION

The invention is further described below in non-limiting examples.

### EXAMPLES

The peptides TF2D-1, TF2D-5 and TF2D-5a have been studied in more detail.

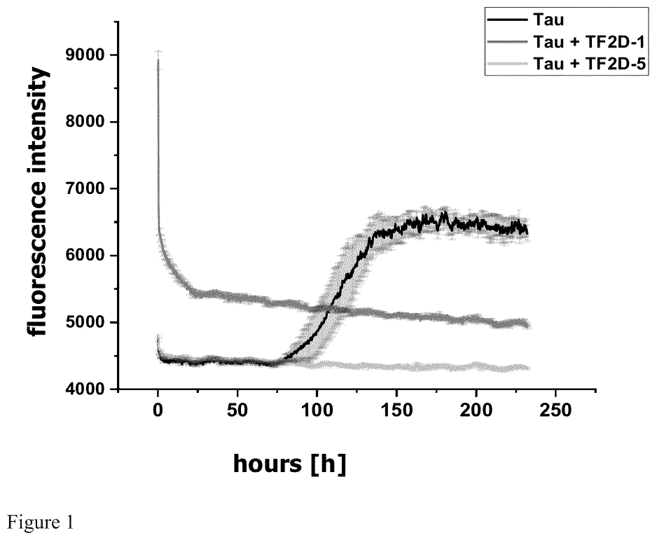

The results are summarized in FIGS. 1, 2a, 2b, 3, 4, 5 and 6.

FIG. 1: TF2D-1 and TF2D-5 inhibit tau aggregation, analyzed by ThT analysis

The effects of TF2D-1 and TF2D-5 on tau aggregation are shown. The ThT assay was performed by incubation of 15 μM tau with 8 μM heparin, 20 μM THT and 15 μM of either of the two peptides TF2D-1 or TF2D-5 at 37° C. in PBS pH 7.4. The sample without addition of the peptide is shown in red. The samples in which TF2D-1 or TF2D-5 was present are shown in green (TF2D-1) and blue (TF2D-5).

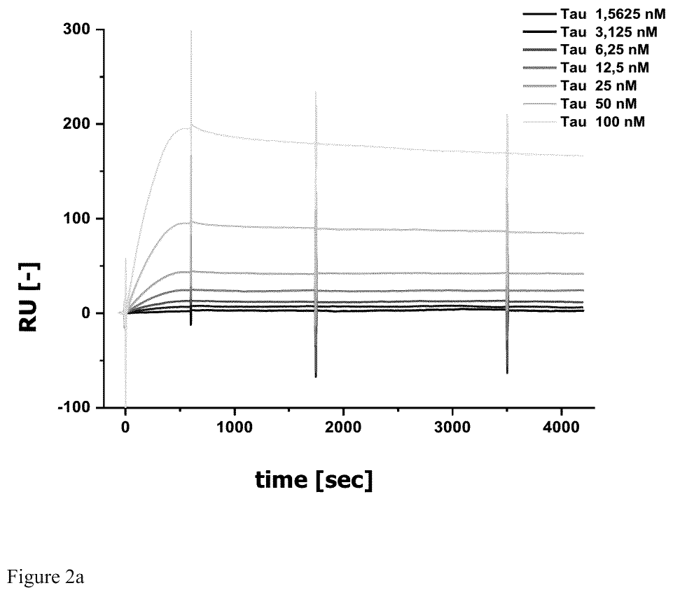

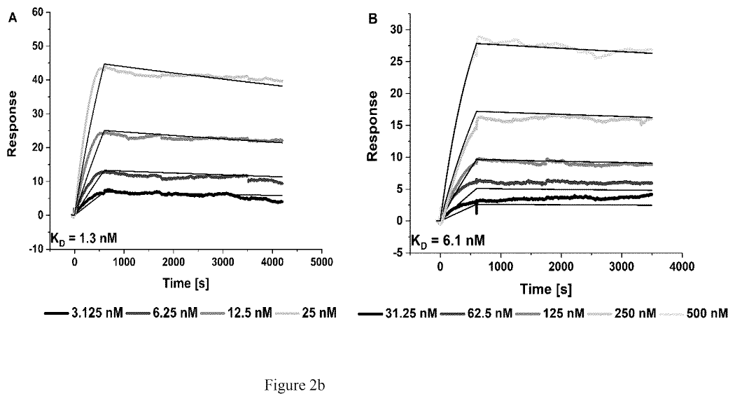

FIG. 2a: TF2D-5 binds with high affinity to tau protein

SPR measurements of TF2D-5 with the analyte tau. Seven concentrations of tau were prepared for the measurement, with the highest concentration at 100 nM and the lowest concentration at 1.56 nM. The running buffer contained TBS with 0.1% Tween. The Koff was determined to be approximately 6×10-5 s-1.

FIG. 2b: TF2D-5 and TF2D-5a bind with high affinity to the tau protein

SPR measurement of TF2D-5 and TF2D-5a with full-length tau 441 A) The binding curves and the calculated fit for the affinity of TF2D-5 to monomeric full-length tau 441 gave a K_{D} of 1.3 nM. The tau concentration during the measurement varied from 3.125 nM to 25 nM. B) The binding affinity of TF2D-5a to tau in full length was determined to be 6.1 nM. Tau was used at concentrations from 31.25 nM to 500 nM. SPR measurements of A and B were recorded on a T200 Biacore instrument, the respective peptide was immobilised on a CM5 chip from Cytiva, while monomeric tau served as the analyte. The tests were performed using TBS pH 7.4, 0.05% Tween 20 and 10 pM DTT as running buffer. The fit calculation was performed using the internal evaluation software of the T200 Biacore. A 1:1 model was used for both fits.

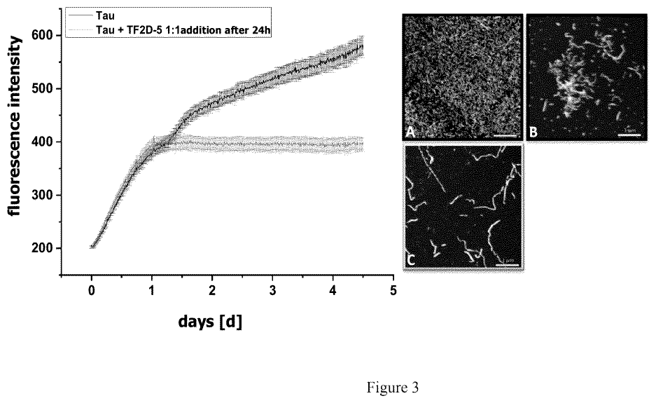

FIG. 3: TF2D-5 inhibits tau aggregation, analyzed by ThT and AFM analysis

Shown are the effects of TF2D-5 on tau aggregation. The ThT assay was performed by incubation of 15 μM tau with 8 μM heparin, 20 μM ThT, 10% DMSO and different concentrations of TF2D-5 at 37° C. pH 7.0. The images next to the graph show the respective AFM measurements of each sample taken after 5 days. A shows the sample that did not contain TF2D-5. 6 shows the sample from A with a dilution of 1:10. The image marked with C corresponds to the blue ThT curve without dilution, where TF2D-5 was added after 24 hours. All AFM measurements were performed by drying 1 μl on a mica, followed by two wash steps with 100 μl water and measurement on JPK Nanowizard3 with intermittent contact air and an OMCL AC160TS as cantilever.

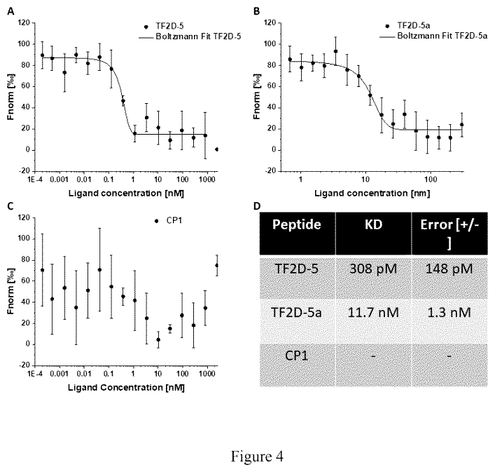

FIG. 4: TF2D-5 and TF2D-5a affinity to tau is sequence specific

MST measurement of TF2D-5, TF2D-5a and CP1 with tau in full-length. A) Boltzmann fit of TF2D-5 MST measurements with tau in full-length. TF2D-5 was labelled with CF633. The concentration of TF2D-5 was 500 nM, while the concentration of tau in full-length was between 2.5 μM and 0.2 pM. The measurement was performed in 1×PBS pH 7.4 with 150 mM NaCl. The calculated K_{D} is 308 pM+/−148 pM. B) Boltzmann fit of TF2D-5a MST measurements with tau in full length. TF2D-5a was labelled with CF633. The concentration of TF2D-5a was 62.5 nM, while the concentration of tau in full-length varied between 300 nM and 0.5 nM. The measurement was performed in 0.1 M sodium phosphate buffer pH 7.4 with 75 mM sodium sulphate and 0.05% Tween 20. The calculated K_{D} is 11.7 nM+/−1.3 nM. C) Average of the CP1-MST measurements with tau in full length. CP1 consists of the same amino acids as TF2D-5, but with a different distribution of the amino acids. CP1 was labelled with CF633. The concentration of CP1 was 62.5 nM, while the concentration of tau in full-length varied between 2.5 M and 0.2 pM. The measurement was performed in 0.1 M sodium phosphate buffer pH 7.4 with 75 mM sodium sulphate and 0.05% Tween 20. No Boltzmann fit and no K_{D} could be calculated for the tested concentration range. D) Calculated K_{D}s of the individual peptides to tau in full-length at a glance. All experiments were repeated four times and the average of all measurements was calculated, which in turn was used to calculate the fits (Boltzmann).

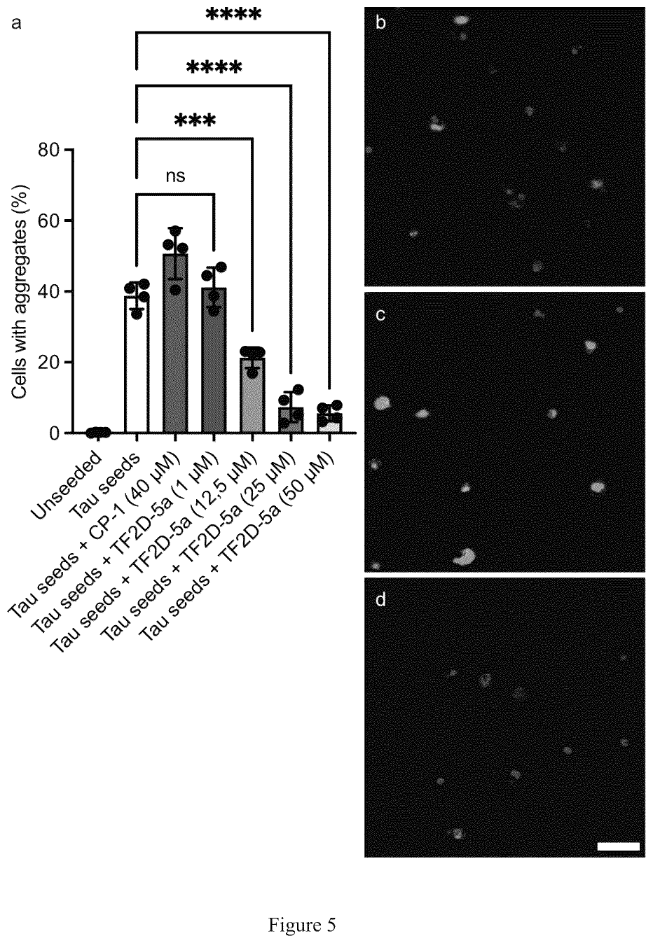

FIG. 5: TF2D-5a inhibits the aggregation of tau in cells.

A) TF2D-5a and the D-peptide CP-1, as negative control, were tested for their ability to inhibit the tau aggregation in tauk18 (LM)-YFP cells. The scale bar corresponds to 50 μm and applies to b-d. The significance was calculated using a one-way ANOVA followed by Dunnett's test for multiple comparisons. Three asterisks represent a p-value of less than 0.001 and four asterisks a p-value of less than 0.0001. B) In contrast to unseeded cells, which had no tau aggregates on the third day after planting out, seeding with sonified tau fibrils resulted in aggregation in about 40% of cells after three days of incubation. C) A three-day treatment with the D-peptide CP-1 as negative control did not inhibit tau aggregation. D) In contrast to that, the treatment with increasing concentrations of TF2D-5a resulted in a concentration-dependent reduction in the number of cells with tau aggregates, which was significant at 12.5 UM and was further reduced at 25 μM and 50 μM TF2D-5a.

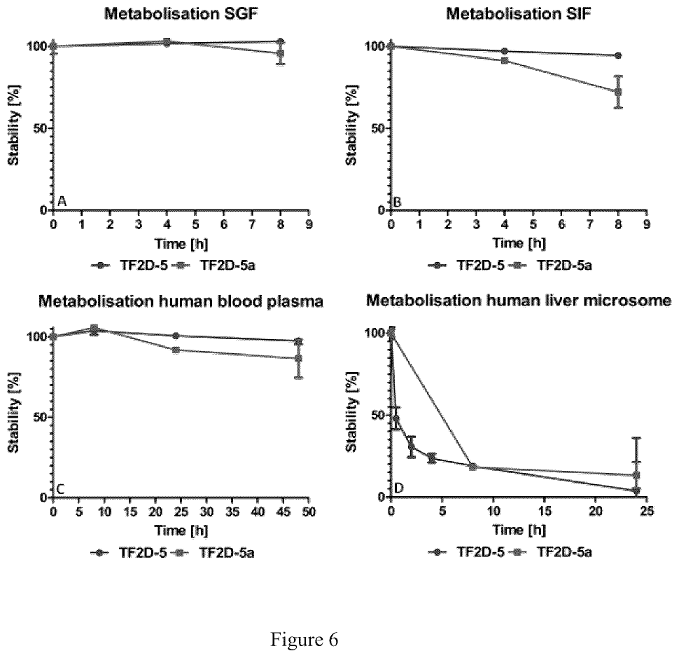

FIG. 6: TF2D-5 and TF2D-5a are very stable in different body fluids

Stability study of TF2D-5 and TF2D-5a in different simulated human body fluids. A) Stability of the peptides in simulated gastric fluid (SGF). The simulated gastric fluid consisted of 2 mg/ml sodium chloride, 3.2 mg/ml pepsin and 80 mM HCl; pH 1. B) Stability of TF2D-5 and TF2D-5a in simulated intestinal fluid (SIF). Simulated intestinal fluid consisted of 6.8 mg/ml potassium dihydrogen phosphate, 10 mg/ml pancreatic powder and 15.4 mM NaOH; pH 6.8. C) Stability of TF2D 5 and TF2D-5a in human blood plasma. Human K3EDTA blood plasma was obtained from BioTrend. D) Stability of TF2D-5 and TF2D-5a in human liver microsomes. Human liver microsomes consisted of 6 mg/ml protein (containing cytochrome P450 (CYP) enzymes), 75 mM sucrose, 1.3 mM NADPH, 3.3 mM D-glucose-6-phosphate, 0.2 u/ml D-glucose-6-phosphate dehydrogenase, 3.3 mM magnesium chloride and 100 mM potassium phosphate pH 7.4.

1.-15. (canceled)

16. A peptide comprising an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7, and homologues, fragments and parts thereof.

17. The peptide of claim 16, wherein the peptide comprises an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7, or homologues having an identity of at least 80% thereof.

18. The peptide of claim 16, wherein a CONH2 group or a COH group, COCl group, COBr group, CONH-alkyl residue or a CONH-alkyl-amine residue is present at a free C-terminus of the peptide instead of a carboxyl group.

19. The peptide of claim 16, wherein the peptide is cyclized.

20. The peptide of claim 16, wherein the peptide comprises from 2 to 10 copies of sequences having SEQ ID NO: 1, SEQ ID NO: 2, SEQ ID NO: 3, SEQ ID NO: 4, SEQ ID NO: 5, SEQ ID NO: 6 or SEQ ID NO: 7.

21. The peptide of claim 16, wherein the peptide comprises more than 10 copies of sequences having SEQ ID NO: 1, SEQ ID NO: 2, SEQ ID NO: 3, SEQ ID NO: 4, SEQ ID NO: 5, SEQ ID NO: 6 or SEQ ID NO: 7.

22. The peptide of claim 16, wherein the peptide consists essentially of D-amino acids.

23. The peptide of claim 16, wherein the peptide consists of an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7.

24. The peptide of claim 16, wherein the peptide is linked to a further substance.

25. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are covalently linked to each other.

26. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are non-covalently linked to each other.

27. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other without a linker.

28. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other with a linker group.

29. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other in a linear manner.

30. The peptide of claim 16, wherein several peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to each other in a branched manner.

31. The peptide of claim 16, wherein the peptide is a dendrimer in which peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to a platform molecule.

32. The peptide of claim 16, wherein the peptide binds to a tau peptide with a K_{D} of less than 1 μM.

33. The peptide of claim 16, wherein the peptide is capable of preventing a formation of tau peptide oligomers and/or tau peptide aggregates.

34. The peptide of claim 16, wherein the peptide is capable of detoxifying tau peptide oligomers and/or tau peptide aggregates.

35. The peptide of claim 16, wherein the peptide is suitable for use in treating tauopathies.
